(19) United States
(12) Patent Application Publication
(10) Pub. No.: US 2020/0046551 A1
(43) Pub. Date: Feb. 13, 2020

(54) Body cooling devices

(71) Applicants: Tenisha Garrett, Warren, MI (US); Santonio Garrett, Warren, MI (US)

(72) Inventors: Tenisha Garrett, Warren, MI (US); Santonio Garrett, Warren, MI (US)

(21) Appl. No.: 16/101,186

(22) Filed: Aug. 10, 2018

(51) Int. Cl. A61F 7/10; A61B 5/0205; A61B 5/00; A61F 7/00

(52) U.S. Cl. CPC A61F 7/10; A61B 5/02055; A61B 5/6824; A61B 5/02438; A61F 7/007; A61B 5/002; A61B 5/0008; A61B 5/6831

(57) ABSTRACT

The invention is directed to body cooling devices. The devices are provided in three embodiments: a headband, a wristband, and an upper armband. The wristband version is worn on one of the user's wrists like a wristwatch or bracelet, and monitors body temperature and pulse rate with sensors. The wristband version further cools the body with cooling elements. The cooling elements and sensors are positioned on the inner surface such that they are in contact with the user's skin. Graph readouts of body temperature and pulse rate are displayed on the outer surface on a curved visual display, and transmitted via Bluetooth 4.0™. The headband version is a scaled-up version of the wristband version, providing the same features, and is worn around the head at the forehead level. The upper armband version provides the same features except the visual display, and further provides a medium range Bluetooth 4.0™transmitter, which may be used to periodically transmit accumulated data to a sports trainer or other person via a mobile application.

### CROSS-REFERENCE TO RELATED APPLICATIONS

This application relates back to, and claims the priority of Provisional Patent Application No. 62/543,913 filed on Aug. 10, 2017.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to mobile electronic devices, and in particular to body cooling devices. The Fitbit™ personal fitness monitor has revolutionized the fitness industry, as millions of users wear these devices on their wrists to monitor such vital signs as the pulse and respiratory rate during exercise. Interest in fitness training is increasing rapidly, particularly among young electronics consumers, and the Fitbit™ has proven to be very popular. However, particularly in warmer Southern states, the danger of heatstroke or heat exhaustion during an outdoor run or workout is a serious concern.

Several devices have been developed to lower the user's body temperature. These have generally focused on patients in a hospital setting. Each has proven to be less than satisfactory in its own way for active, highly mobile users.

Active body cooling with vasodilation to reduce body temperature, U.S. Pat. No. 7,361,186 (priority Aug. 25, 2003), provides a system for the cooling of a patient's body. Therapeutic induced mild-moderate hypothermia can be beneficial for people suffering stroke, myocardial infarction, cardiac arrest and other conditions involving reduced blood supply. A non-invasive, aggressive heat transfer technique for lowering body temperature is to externally cool the exterior surface of the patient's body. Such exterior surface cooling could be achieved, for example by direct contact with a cooling fluid, such as by immersing the patient's body in the cooling fluid or by directing the flow of the cooling fluid around the patient's body. Another technique for external surface cooling is to apply a contact-cooling pad to the exterior surface of the patient and to circulate a cooling fluid, such as water or an aqueous solution, through the contact pad to cool the patient. The exterior surface cooling is accompanied by administration to the patient of a vasodilation drug at a time to promote vasodilation, that corresponds with some or all of the aggressive heat transfer. Aggressive heat transfer from the exterior body surface rapidly removes thermal energy from near-surface regions of the patient's body. The vasodilation drug promotes dilation of blood vessels in the near surface region and, consequently, increased flow of blood through those blood vessels to assist more rapid cooling of other areas of the body.

Method and apparatus for reducing body temperature of a subject, U.S. Pat. No. 7,621,945 (priority Apr. 29, 2002), provides a cooling system which includes a console and a tissue cooling device such as a head-cooling device. An operator applies the head-cooling device to the head of a patient at risk for ischemic injury. The console provides a cooling fluid to a fluid circulation space located between the cooling device and the patient's head under a positive gage pressure. Direct contact between the cooling fluid and the patient's head provides a relatively rapid induction of systemic hypothermia in the patient, thereby minimizing or preventing ischemic injury in the patient. The console also removes air from a channel disposed about an inner rim of the cooling device, using a negative gage pressure. Such removal of the air from the channel seals the rim of the cooling device to the head of the patient, including portions of the channel in contact with hair of the patient's head, and minimizes leaking of the cooling fluid beyond the rim of the cooling device.

Selective organ cooling catheter with guidewire apparatus and temperature-monitoring device, U.S. Pat. No. 6,312,452 (priority Jan. 23, 1998), provides a guidable catheter for heating or cooling a surrounding fluid in a feeding vessel in a vasculature of a patient. The catheter includes a heat transfer element, the heat transfer element having a plurality of exterior surface irregularities shaped and arranged to create turbulence in a surrounding fluid. The surface irregularities have a depth at least equal to the boundary layer thickness of flow of the surrounding fluid in the feeding vessel. The catheter assembly also includes a supply catheter having a portion disposed within the heat transfer element to deliver a working fluid to an interior of the heat transfer element. The catheter assembly further includes a return catheter to return a working fluid from the interior of the heat transfer element. A guidewire tube is provided adjacent one of the supply catheter or the return catheter and runs substantially parallel to the axis of the guidable catheter to receive a guidewire disposed within the guidewire tube. The guidable catheter may further have a temperature-monitoring device disposed at the distal tip of the guidewire. The temperature-monitoring device may be a thermocouple or a thermistor. Feedback may further be provided to control the temperature of a source of working fluid. A method is also provided for selectively controlling the temperature of a selected volume of blood in a patient. The method includes introducing a guidewire into a blood vessel feeding a selected volume of blood in a patient and introducing a catheter assembly into the blood vessel feeding a selected volume of blood in a patient by inserting the guidewire into a guidewire tube in the catheter assembly. A working fluid is delivered from a source of working fluid through a supply catheter in the catheter assembly and returned through a return catheter in the catheter assembly. Heat is transferred between a heat transfer element forming a distal end of the catheter assembly and the volume of blood in the feeding vessel. The temperature is monitored of the volume of blood in the feeding vessel by measuring the temperature with a temperature-monitoring device disposed at or near the distal tip of the guidewire.

Cooling devices with high-efficiency cooling features, U.S. Pat. No. 6,718,785 (priority Apr. 11, 2000), provides Cooling devices to reduce a person's temperature by evaporative, convective, and/or conductive cooling. One such device maximizes evaporative cooling by aiding the flow of air to the person and the removal of vapor-laden air from the person. An upper sheet and a base sheet are adhered to define numerous elongated, parallel, inflatable cooling chambers separated by flat connecting membranes. Ventilating cross-members interconnect the cooling chambers. Air enters the chambers through an inlet, exits the chambers toward the person through air permeable regions of the base sheet. Air heated by the person's body exits the device upward through evaporation openings in the connecting membranes. The foregoing device, or different variations thereof, may be modified for use in conductive cooling by adding an absorbent sheet beneath the base sheet, or substituting the absorbent sheet for the base sheet itself. This device directs air upon the wetted absorbent sheet to cool this layer, and thereby conductively cool the patient's skin in thermal contact with the absorbent sheet. As one example, this device may be configured in serpentine shape, with multiple winding segments. The device may include body-contour slits extending inward from the perimeter, permitting the device to conform to a person's legs and outstretched arms. Cooling devices may also include optional features to enhance thermal contact between the absorbent sheet and the person's skin, and/or to prevent water runoff from the cooling field.

Cooling device and method, U.S. Patent Appl. No. 2007/0193278A1 (priority Feb. 16, 2006), provides a device for providing cooling to a body. The device operates in response to a measured change in temperature of the body's skin surface over a predetermined period of time or manually in response to a perceived or anticipated change in body temperature by the user. The device includes a controller, a temperature sensor, a power source, a thermoelectric cooler, and a heat absorbing reservoir containing a phase change material. The thermoelectric cooler has a heat absorbing surface engageable with the body to be cooled and a heat rejecting surface engaged with the heat absorbing reservoir. The controller is operatively connected to the temperature sensor, the power source, and the thermoelectric cooler and activates the cooler when the temperature sensor provides signals indicative of a predetermined rate of temperature change or in manual mode by user. A method of treating hot flashes with the device is also disclosed.

Devices which may be worn on various parts of the body, which provide a cooling effect to comfortably reduce body temperature during such activities and measure and transmit data about the user's body temperature, would be well received. Body cooling devices, which may be worn on the forehead, as an armband on one upper arm, and on one wrist to effectively and comfortably monitor and reduce body temperature, would resolve these problems.

### SUMMARY OF THE INVENTION

Accordingly, the invention is directed to body cooling devices. The devices are provided in three embodiments: a headband, a wristband, and an upper armband. The wristband version is worn on one of the user's wrists like a wristwatch or bracelet, and monitors body temperature and pulse rate with sensors. The wristband version further cools the body with cooling elements. The cooling elements and sensors are positioned on the inner surface such that they are in contact with the user's skin. Graph readouts of body temperature and pulse rate are displayed on the outer surface on a curved visual display, and transmitted via Bluetooth 4.0™. The headband version is a scaled-up version of the wristband version, providing the same features, and is worn around the head at the forehead level. The upper armband version provides the same features except the visual display, and further provides a medium range Bluetooth 4.0™ transmitter, which may be used to periodically transmit accumulated data to a sports trainer or other person via a mobile application.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate three embodiments of the invention and, together with the description, serve to explain the principles of the invention.

FIG. 1 is a top view of the first exemplary embodiment, displaying the wristband 10, and the visual display 11.

FIG. 2 is a front view of the first exemplary embodiment, displaying the wristband 10.

FIG. 3 is a side perspective view of the first exemplary embodiment, displaying the wristband 10, and the visual display 11.

FIG. 4 is a top perspective view of the first exemplary embodiment, displaying the wristband 10, and the visual display 11.

FIG. 5 is a side view of the second exemplary embodiment, displaying the visual display 11, and the headband 12.

FIG. 6 is a front view of the second exemplary embodiment, displaying the headband 12.

FIG. 7 is a top view of the second exemplary embodiment, displaying the visual display 11, and the headband 12.

FIG. 8 is a side view of the third exemplary embodiment, displaying the upper armband 13, and the medium range transceiver 14.

FIG. 9 is a front view of the third exemplary embodiment, displaying the upper armband 13.

FIG. 10 is a top view of the third exemplary embodiment, displaying the upper armband 13, and the medium range transceiver 14.

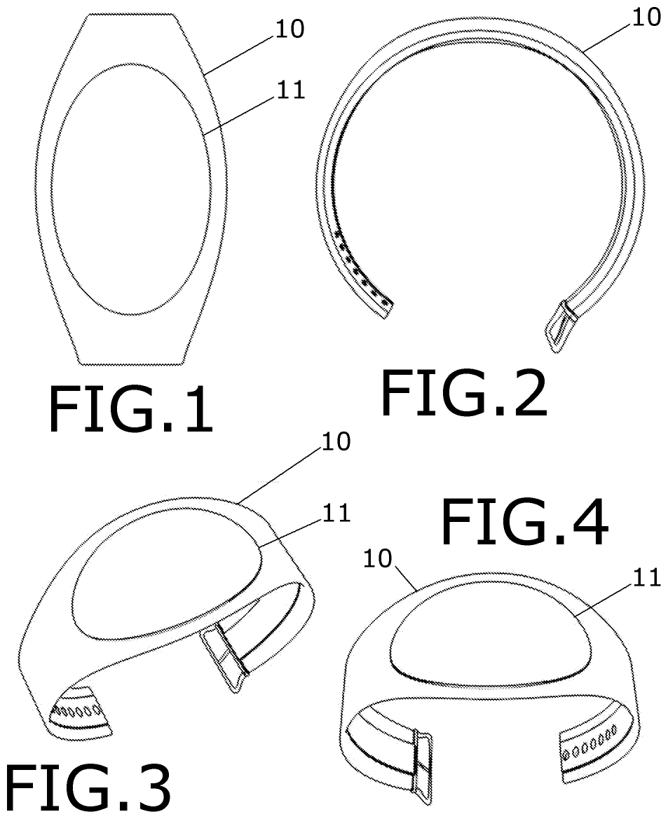

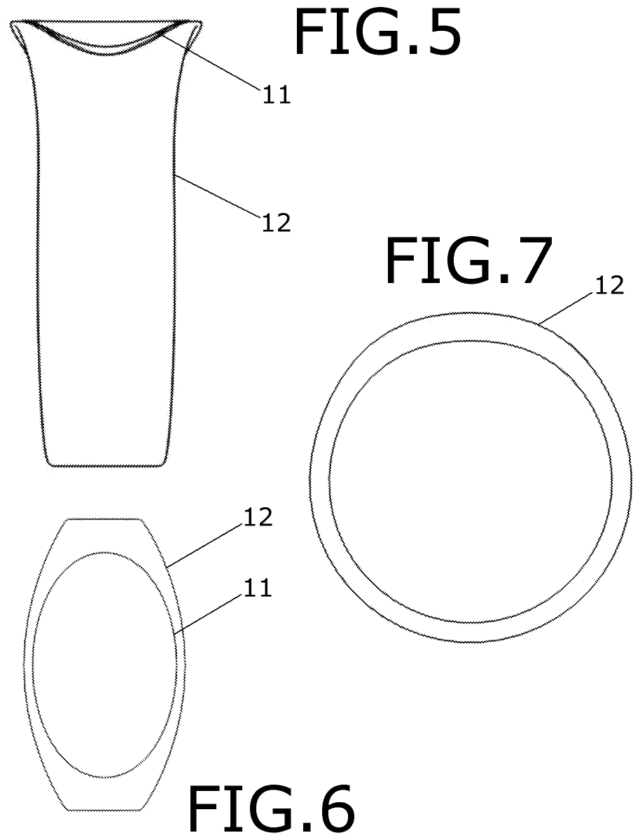

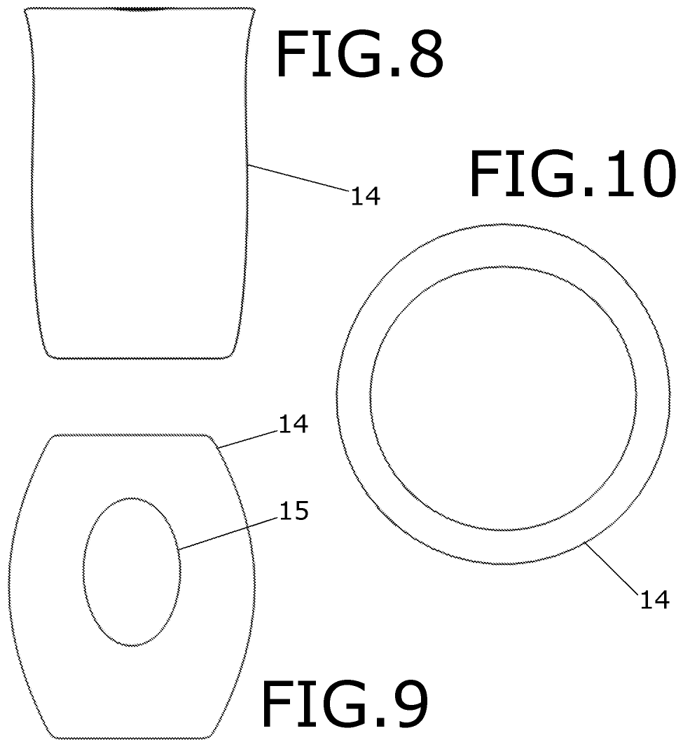

### DETAILED DESCRIPTION OF THE INVENTION

Referring now to the invention in more detail, the invention is body cooling devices.

The first exemplary embodiment is comprised of a wristband 10 which is intended to be worn on one of the user's wrists, much like a wristwatch or bracelet. The wristband 10 provides a curved and contoured visual display 11. The wristband 10 further provides cooling elements and sensors, which gather data on the user's body temperature and pulse rate, on the inner surface such that they are in contact with the user's skin when worn. Internally, the wristband 10 further provides a short range transmitter. The wristband 10 is battery powered.

The second exemplary embodiment is identical in structure and function to the first exemplary embodiment, with the following modifications. The second exemplary embodiment is a scaled-up version such that it may be worn as a headband 12, which is intended to be worn around the user's head above eyebrow level.

The third exemplary embodiment is identical in structure and function to the first exemplary embodiment, with the following modifications. The third exemplary embodiment is a wider version which is intended to be worn on one of the user's arms above the elbow, as an upper armband 13. Instead of a visual display, the upper armband 13 provides a medium range transceiver 14.

The short range transmitters of the wristband 10 and the headband 12, and the medium ranged transceiver 14 of the upper armband 13, are synchronized with each other and operate on Bluetooth 4.0™ or a similar medium range communications protocol. The short range transmitters transmit the body temperature and pulse rate data acquired in real time to the medium range transceiver 14. The medium range transmitter 14 transmits this data, plus similar data acquired by the upper armband 13, to a sports trainer, medical caregiver, or other authorized person via a mobile application which retrieves and stores the data. All transmissions are encrypted.

To use the first, second, and third exemplary embodiments, the user may secure the devices to their respective positions on the user's body. Further operation of the devices is automatic.

The devices are preferably manufactured from rigid, durable materials which may provide a decorative element, such as plastic, stainless steel, brass, malacrylate, aluminum alloy, copper alloy, acrylic polymer, and ceramics. Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. Body cooling devices which provide a medical monitoring function, comprised of a wristband which is intended to be worn on one of the user's wrists, much like a wristwatch or bracelet, providing a curved and contoured visual display, and further provides cooling elements, which either provide electrically-powered cooling or are filled with a freezable gel, and sensors, which gather data on the user's body temperature and pulse rate, on the inner surface such that they are in contact with the user's skin when worn.

2. The body cooling devices of claim 1, wherein internally, the wristband further provides a short range battery-powered transmitter.

3. The body cooling devices of claim 1, wherein the devices further provide a headband, which is intended to be worn around the user's head above eyebrow level.

4. The body cooling devices of claim 1, wherein the devices further provide a wider band which is intended to be worn on one of the user's arms above the elbow, as an upper armband, providing a medium range transceiver instead of a visual display.

5. The body cooling devices of claim 4, wherein the short range transmitters of the wristband and the headband, and the medium ranged transceiver of the upper armband, are synchronized with each other and operate on Bluetooth 4.0™ or a similar medium range communications protocol.

6. The body cooling devices of claim 4, wherein the short range transmitters transmit the body temperature and pulse rate data acquired in real time to the medium range transceiver.

7. The body cooling devices of claim 4, wherein the medium range transmitter transmits received data, plus similar data acquired by the upper armband, to a sports trainer, medical caregiver, or other authorized person in an encrypted form via a mobile application which retrieves and stores the data.

8. The body cooling devices of claim 4, wherein the user may secure the devices to their respective positions on the user's body.
